(19) United States
(12) Patent Application Publication
(10) Pub. No.: US 2004/0214760 A1
(43) Pub. Date: Oct. 28, 2004

(54) Inhibition pf protein-phosphatases for the treatment of heart failure

(76) Inventors: Ramesh Gupta, Rochester Hills, MI (US); Hani Sabbah, Waterford, MI (US)

Correspondence Address: Kenneth I Kohn, Kohn & Associates, Suite 410, 30500 Northwestern Highway, Farmington Hills, MI, 48334, US

(21) Appl. No.: 10/450,995

(22) PCT Filed: Jan. 15, 2002
(86) PCT No.: PCT/US02/00957

Related U.S. Application Data
(60) Provisional application No. 60/261,931, filed on Jan. 15, 2001.

(51) Int. Cl. A61K 38/50

(52) U.S. Cl. 514/12

(57) ABSTRACT

There is provided a method for treating an individual with cardiovascular disease, including the step of administering a therapeutically effective amount of protein phosphatase inhibitor and analogues thereof, to the individual after the onset of cardiac ischemia. Also provided is a method of treating an individual with cardiovascular disease by selectively inhibiting protein phosphatase activity in the heart. A composition for the treatment of heart failure, the composition including an inhibitor of PP1 and a pharmaceutically acceptable carrier is also provided. There is provided a sequence encoding inhibitor-2.

### BACKGROUND OF THE INVENTION

1. Technical Field

The present invention relates to a composition and treatment for heart failure. More specifically, the present invention relates to protein-phosphatases inhibitors for use in treating heart failure.

2. Description of Related Art

A practical and entirely pragmatic definition of heart failure is that heart failure is a clinical syndrome (readily diagnosed by doctors) caused by an abnormality of the heart and recognized by a characteristic pattern of hemodynamic, renal, neural, and hormonal responses. This definition requires an abnormality of the heart to be present and states that much of the clinical picture is a consequence of the response of the body to the malfunction of the heart.

Many adjectives have been used to modify the description of heart failure, including high and low out-put cardiac failure, forward and backward failure, right and left heart failure, congestive heart failure, systolic and diastolic heart failure, and acute and chronic heart failure. For practical purposes patients can be classified using three categories: acute heart failure (pulmonary edema), circulatory collapse, and chronic heart failure.

Chronic heart failure should not be regarded as a steady-state condition, as the function of the heart and the interaction of the heart and circulation vary with time. Many patients with chronic heart failure develop acute exacerbations that are not always due to identifiable causes, such as arrhythmias, ischemic episodes, failure to take medicines, pulmonary embolus, dietary indiscretion, lung infection, or concurrent illness. The clinical entity whereby patients spontaneously recover and relapse with chronic heart failure can be called undulating heart failure and is not yet fully understood. The terms “right” and “left” heart failure are widely used terms that convey helpful clinical information between doctors but are otherwise misleading, as the commonest cause of “right” heart failure is failure of the left ventricle. In so-called high output cardiac failure the primary abnormality is not one of ventricular dysfunction, the increased cardiac function is a response to systemic metabolic or circulatory changes. These conditions, such as nephritis, Paget's disease, arteriovenous shunts, thyrotoxicosis, pregnancy, anemia, and beri-beri, are perhaps best regarded as conditions of salt and water retention rather than heart failure.

In Western countries, myocardial infarction is among the most common diagnoses in hospitalized patients. In the United States, approximately 1.5 million myocardial infarctions (MIs) occur each year, and mortality with acute infarction is approximately 30 percent (Pasternak, R. and Braunwald, E., Acute Myocardial Infarction, HARRISON'S PRINCIPLES OF INTERNAL MEDICINE, 13th Ed., McGraw Hill Inc., p.p. 1066-77 (1994)). More than half of the deaths that result from myocardial infarction occur before the patient reaches the hospital, and an additional 5-10% of survivors die in the first year (Pasternak, R. C. and Braunwald, E. Acute Myocardial Infarction, HARRISON'S PRINCIPLES OF INTERNAL MEDICINE, 13th Ed., McGraw Hill Inc., p.p. 1066-77 (1994)).

Myocardial infarction occurs generally with an abrupt decrease in coronary blood flow that follows a thrombotic occlusion of a coronary artery. The occluded artery often has been narrowed previously by atherosclerosis, and the risk of recurrent nonfatal myocardial infarction persists in many patients. Ultimately, the extent of myocardial damage caused by the coronary occlusion depends upon the “territory” supplied by the affected vessel, the degree of occlusion of the vessel, the amount of blood supplied by collateral vessels to the affected tissue, and the demand for oxygen of the myocardium whose blood supply has suddenly been limited (Pasternak, R. and Braunwald, E. Acute Myocardial Infarction, HARRISON'S PRINCIPLES OF INTERNAL MEDICINE, 13th Ed., McGraw Hill Inc., p.p. 1066-77 (1994)).

Because acute myocardial infarction frequently results in death, scientists and physicians have been studying the effects of myocardial ischemia for many years. It is hoped that, through better understanding of the processes involved in myocardial infarction, methods to minimize the deleterious effects produced by an abrupt decrease in myocardial blood flow can be developed. However, since the onset of a myocardial infarction usually cannot be predicted, the ideal treatment regime would be one that is effective when administered after the onset of the infarction process. Developing treatments that limit damage to the myocardium after the initiation of the infarction process poses a tremendous challenge.

The prognosis in acute myocardial infarction is largely related to the extent of mechanical (“pump” failure of the heart) or electrical (arrhythmia) complications. Ventricular fibrillation is the most common cause of arrhythmic-failure, with death frequently occurring before the patient can reach a hospital. However, pump failure is the primary cause of in-hospital death from acute myocardial infarction. There is a strong correlation between the degree of pump failure, the extent of ischemic necrosis, and mortality (Pasternak, R. and Braunwald, E., Acute Myocardial Infarction, HARRISON'S PRINCIPLES OF INTERNAL MEDICINE, 13th Ed., McGraw Hill Inc., p.p. 1066-77 (1994)).

An important development in the care of patients that suffer from an acute myocardial infarction is the use of pharmacologic or mechanical techniques to induce early reperfusion of the ischemic myocardium. Such techniques can “salvage” the tissue before it becomes damaged irreversibly. Since most acute myocardial infarctions are caused by thrombotic occlusion, thrombolytic agents (e.g. tissue plasminogen activator, streptokinase, and an isolated plasminogen streptokinase activator complex) can often restore coronary artery flow. Blood flow also can be restored mechanically with primary percutaneous transluminal coronary angioplasty.

Percutaneous transluminal coronary angioplasty is effective in restoring perfusion in acute myocardial infarction without having to use thrombolysis, and can be slightly more effective than present pharmacologic therapy. Still, percutaneous transluminal coronary angioplasty is expensive, requires highly trained personnel, and is limited seriously by facility requirements and other logistic considerations.

The clinical success achieved with percutaneous transluminal coronary angioplasty and thrombolytic agents has instigated a search for other mechanisms to limit the extent of ischemic damage. Of particular value would be the development of pharmacologic agents that delay the onset of cell death under ischemic conditions, compounds that enhance the survival of tissues after an ischemic episode, and/or drugs that diminish cell injury associated with reestablishment of blood flow or reperfusion. Such agents, used alone, should limit infarction size; however, they can be even more useful when employed as an adjunct to thrombolytic or percutaneous transluminal coronary angioplasty therapy.

With the exception of percutaneous transluminal coronary angioplasty and thrombolytic therapy, there are few indications that procedures to reduce the size of ischemic damage can be developed. However, the study of Murry et al., Circulation 74:1124-36 (1986), demonstrated that a significant amount of the myocardium that normally infarcts following a coronary occlusion in dogs could be salvaged if the artery was subjected first to controlled, brief occlusions, and then reperfused prior to the prolonged, myocardial infarction-causing occlusion. This phenomenon, referred to as ischemic preconditioning, was subsequently reported to occur in rabbits, pigs, rats and isolated hearts (Cohen M., et al., Cardiol. Rev. 3(3):137-49 (1995)). Claims that preconditioning has beneficial effects in humans have also been made (Deutsch, et al., Circulation, 82:2044-51 (1990); and Yellon, et al., Lancet, 342:276-77 (1993)), resulting in investigations to determine the biochemical mechanism(s) by which preconditioning leads to protection.

Despite the above methodology, the prior art is deficient in the identification of pharmacological agents that can diminish myocardial infarction and delay cell injury or death in ischemic cardiac tissue after the onset of myocardial infarction.

Another approach considered is the use of compounds which affect protein phosphorylation. Many eukaryotic cell functions, including signal transduction, cell adhesion, gene transcription, RNA splicing, apoptosis and cell proliferation, are controlled by protein phosphorylation. Protein phosphorylation is in turn regulated by the dynamic relationship between kinases and phosphatases. Considerable research in synthetic chemistry has focused on protein kinases. However, recent biological evidence for multiple regulatory functions of protein phosphatases has triggered further investigation of phosphatases. The protein phosphatases represent unique and attractive targets for small-molecule inhibition and pharmacological intervention.

Phosphatases remove phosphate groups from molecules previously activated by kinases and control most cellular signaling events that regulate cell growth and differentiation, cell-to-cell contacts, the cell cycle and oncogenesis. Protein phosphorylation is the ubiquitous strategy used to control the activities of eukaryotic cells. It is estimated that more than 1000 of the 10,000 proteins active in a typical mammalian cell are phosphorylated. In phosphorylation, the high energy phosphate which confers activation is transferred from adenosine triphosphate molecules to a protein by protein kinases, and is subsequently removed from the protein by protein phosphatases.

There appears to be three evolutionarily-distinct protein phosphatase gene families: protein phosphatases (PPs); protein tyrosine phosphatases (PTPs); and acid/alkaline phosphatases (APs). PPs dephosphorylate phosphoserine/threonine residues and are an important regulator of many cAMP-mediated hormone responses in cells. PTPs reverse the effects of protein tyrosine kinases and play a significant role in cell cycle and cell signaling processes. APs dephosphorylate substrates in vitro, although their role in vivo is not well known.

PPs can be cytosolic or associated with a receptor and can be separated into four distinct groups: PP-I, PP-IIA, PP-IIB, and PP-TIC. (Cohen, P. (1989) Annu. Rev. Biochem. 58:453-508.) PP-IIC is a relatively minor phosphatase that is unrelated to the other three. The three principle PPs are composed of a homologous catalytic subunit coupled with one or more regulatory Subunits. PP-I dephosphorylates many of the proteins phosphorylated by cylic AMP-dependent protein kinase (PKA) and is an important regulator of many cyclic AMP-mediated hormone responses in cells. PP-IIA has broad specificity for control of cell cycle, growth, and proliferation, and DNA replication, and is the main phosphatase responsible for reversing the phosphorylations of serine/threonine kinases. PP-IIB, or calcineurin (Cn), is a Ca⁺² activated phosphatase and is particularly abundant in the brain.

PTPs remove phosphate groups from selected phosphotyrosines on particular types of proteins. In so doing, PTPs reverse the effects of protein tyrosine kinases (PTK) and play a significant role in cell cycle and cell signaling processes. (Charbonneau, H. and Tonks, N. K. (1992) Annu. Rev. Cell Biol. 8:463-493.) PTPs possess a high specific enzyme activity relative to their PTK counterparts. In the process of cell division, for example, a specific PTP (M-phase inducer phosphatase) plays a key role in the induction of mitosis by dephosphorylating and activating a specific PTK (CDC2) leading to cell division. (Krishna, S. et al. (1990) Proc. Natl. Acad. Sci. 87:5139-5143.) Tyrosine phosphorylations are therefore short lived and uncommon in resting cells.

Many PTKs are encoded by oncogenes, and it is well known that oncogenesis is often accompanied by increased tyrosine phosphorylation activity. It is therefore possible that PTPs can serve to prevent or reverse cell transformation and the growth of various cancers by controlling the levels of tyrosine phosphorylation in cells. This is supported by studies showing that overexpression of PTP can suppress transformation in cells and that specific inhibition of PTP can enhance cell transformation. (Charbonneau and Tonks, supra.)

PTPs are found in transmembrane, receptor-like and nontransmembrane, non-receptor forms, and are diverse in size (from 20 kDa to greater than 100 kDa) and structure. All PTPs share homology within a region of 240 residues which delineates the catalytic domain and contains the common sequence VHCXAGXXR near the carboxy terminus. The combination of the catalytic domain with a wide variety of structural motifs accounts for the diversity and specificity of these enzymes. In nonreceptor isoforms, noncatalytic sequences can also confer different modes of regulation and target PTPs to various intracellular compartments.

The human protein phosphatase 1 (PP1) enzyme complex has been shown to comprise at least three isoforms of the catalytic subunit: PP1C-alpha, PP1 C-beta, and PP1 C-gamma encoded by different genes. All three subtypes of PP1C exhibit a wide tissue distribution and they all bind to the glycogen associated targeting subunit of PP1. The human G-subunit is probably encoded by a single gene and as determined for rabbit PP1 G-subunit it is expressed in skeletal, heart and diaphragm muscle tissues. A different subtype of PP1-G is expressed in liver. The rabbit skeletal muscle PP1-G has been shown to undergo in vivo and in vitro phosphorylation at several serine residues most of which are located near the NH₂-terminus. Cyclic AMP-dependent protein kinase phosphorylates PP1 G-subunit at Ser⁴⁶ (site 1) and Ser⁶⁵ (site 2). Phosphorylation of site 2 promotes dissociation of the C-subunit and its translocation from the glycogen-protein particles to the cytosol, where it is likely to be inactivated by a cytosolic protein termed inhibitor-1. Thus, phosphorylation of PP1 G-subunit by cyclic AMP-dependent protein kinase results in an immediate inhibition of glycogen synthesis and a stimulation of glycogenolysis. Insulin stimulates glycogen synthesis and inhibits glycogenolysis in skeletal muscle and this is thought to be mediated by the activation of PP1G as a result of the phosphorylation of site 1 on the G-subunit catalyzed by an insulin stimulated protein kinase. The latter was subsequently identified as the Rsk-2 isoform of ribosomal S6 kinase and was also shown to inactivate glycogen synthase kinase-3 (GSK-3) in vitro. Since GSK-3 phosphorylates the sites in glycogen synthase which are dephosphorylated in response to insulin, inhibition of GSK-3 by this hormone, which has been demonstrated in vivo, can also contribute to the activation of glycogen synthesis. A further complication is that GSK-3 phosphorylates the PP1 G-subunit at Ser³⁸ and Ser⁴² in vitro, but the relevance of this to insulin action has still to be evaluated.

The glycogen-associated form of protein phosphatase 1 (PP1 G-subunit) derived from skeletal muscle is a heterodimer composed of a 37 kDa catalytic subunit (C) and a 124 kDa targeting and regulatory subunit (G). PP1-G not only binds to muscle glycogen with high affinity and thus enhances dephosphorylation of glycogen bound PP1 substrates such as glycogen synthase and glycogen phosphorylase kinase but also plays an essential role in the control of glycogen metabolism by different hormones. Phosphorylation at Ser⁴⁶ (site 1) of the G-subunit in response to insulin enhances the activity of PP1-G towards glycogen bound substrates (stimulation of glycogen synthesis and inhibition of glycogenolysis) while phosphorylation at Ser⁶⁵ (site 2) of PP1-G in response to adrenaline causes dissociation of PP1C from the targeting G-subunit thereby inhibiting glycogen synthesis and stimulating glycogenolysis.

In subsets of patients with widespread disorders like obesity, non-insulin dependent diabetes mellitus (NIDDM), essential hypertension, dyslipidemia, and premature atherosclerosis, impaired insulin stimulated non-oxidative glucose disposal, which primarily reflects insulin resistance of skeletal muscle glycogen synthesis, has repeatedly been reported. Resistance to the action of insulin on muscle glycogen synthesis has therefore been proposed as the inherited basis for subsets of disorders in the insulin resistance syndrome. In support of this hypothesis, defective insulin mediated activation of muscle glycogen synthase has been found in glucose tolerant but insulin resistant first degree relatives of Caucasian NIDDM patients. Also in severely insulin resistant Pima Indians a reduced basal and insulin stimulated activity of protein phosphatase 1 in muscle tissue has been demonstrated, providing a mechanism by which glycogen synthase activation by insulin is reduced in these subjects.

It would therefore be useful to develop a composition and method of treating heart disease utilizing inhibitors of protein phosphatases.

### SUMMARY OF THE INVENTION

According to the present invention, there is provided a method for treating an individual with cardiovascular disease, including the step of administering a therapeutically effective amount of protein phosphatase inhibitor and analogues thereof, to the individual after the onset of cardiac ischemia. Also provided is a method of treating an individual with cardiovascular disease by selectively inhibiting protein phosphatase activity in the heart. A composition for the treatment of heart failure includes an inhibitor of PP1 and a pharmaceutically acceptable carrier is also provided. There is provided a sequence encoding inhibitor-2.

### BRIEF DESCRIPTION OF THE DRAWINGS

Other advantages of the present invention are readily appreciated as the same becomes better understood by reference to the following detailed description when considered in connection with the accompanying drawings wherein:

FIG. 1 is a photograph showing the presence of 1-2;

FIG. 2 is a photograph showing the presence of the presence of 1-2 insert confirmed by HindIII restriction enzyme digestion;

FIGS. 3A and B are protein and cDNA sequences of Inhibitor-2, FIG. 3A is Dog LV myocardium and FIG. 3B is Human LV myocardium;

FIG. 4 is a photograph showing an increase in IPTG protein expression;

FIG. 5 is a graph showing percent PP1 activity versus induction time; and

FIG. 6 is a graph showing percent PP1 activity version protein.

### DESCRIPTION OF THE INVENTION

Generally, the present invention provides a composition and treatment for heart failure. The composition and treatment function by inhibiting protein-phosphatases. Specifically, the composition of the present invention functions by inhibiting type-1 protein phosphatase (PP1) activity in the heart. The present invention provides a composition including therein inhibitor-2 (I-2) for inhibiting the PP1. The I-2 can be synthetic or isolated from a mammal having the inhibitor.

“I-2” as used herein, refers to the amino acid sequences of substantially purified I-2 obtained from any species, particularly a mammalian species, including bovine, ovine, porcine, murine, equine, and preferably the human species, from any source, whether natural, synthetic, semi-synthetic, or recombinant.

In “allele” or an “allelic sequence,” as these terms are used herein, is an alternative form of the gene encoding I-2. Alleles can result from at least one mutation in the nucleic acid sequence and can result in altered mRNAs or in polypeptides whose structure or function can or can not be altered. Any given natural or recombinant gene can have none, one, or many allelic forms. Common mutational changes which give rise to alleles are generally ascribed to natural deletions, additions, or substitutions of nucleotides. Each of these types of changes can occur alone, or in combination with the others, one or more times in a given sequence.

“Altered” nucleic acid sequences encoding I-2, as described herein, include those sequences with deletions, insertions, or substitutions of different nucleotides, resulting in a polynucleotide the same I-2 or a polypeptide with at least one functional characteristic of I-2. Included within this definition are polymorphisms which can or can not be readily detectable using a particular oligonucleotide probe of the polynucleotide encoding I-2, and improper or unexpected hybridization to alleles, with a locus other than the normal chromosomal locus for the polynucleotide sequence encoding I-2. The encoded protein can also be “altered,” and can contain deletions, insertions, or substitutions of amino acid residues which produce a silent change and result in a functionally equivalent I-2. Deliberate amino acid substitutions can be made on the basis of similarity in polarity, charge, solubility, hydrophobicity, hydrophilicity, and/or the amphipathic nature of the residues, as long as the biological or immunological activity of I-2 is retained. For example, negatively charged amino acids can include aspartic acid and glutamic acid, positively charged amino acids can include lysine and arginine, and amino acids with uncharged polar head groups having similar hydrophilicity values can include leucine, isoleucine, and valine; glycine and alanine; asparagine and glutamine; serine and threonine; and phenylalanine and tyrosine.

The terms “amino acid” or “amino acid sequence,” as used herein, refer to an oligopeptide, peptide, polypeptide, or protein sequence, or a fragment of any of these, and to naturally occurring or synthetic molecules. In this context, “fragments”, “immunogenic fragments”, or “antigenic fragments” refer to fragments of I-2 which are preferably about 5 to about 15 amino acids in length and which retain some biological activity or immunological activity of I-2. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

“Amplification,” as used herein, relates to the production of additional copies of a nucleic acid sequence. Amplification is generally carried out using polymerase chain reaction (PCR) technologies well known in the art. (See, e.g., Dieffenbach, C. W. and G. S. Dveksler (1995) PCR Primer, a Laboratory Manual, Cold Spring Harbor Press, Plainview, N.Y., pp. 1-5.)

As used herein, the term “antibody” refers to intact molecules as well as to fragments thereof, such as Fa, F(ab′)₂, and Fv fragments, which are capable of binding the epitopic determinant. Antibodies that bind PP1 polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antisense,” as used herein, refers to any composition containing a nucleic acid sequence which is complementary to a specific nucleic acid sequence. The term “antisense strand” is used in reference to a nucleic acid strand that is complementary to the “sense” strand. Antisense molecules can be produced by any method including synthesis or transcription. Once introduced into a cell, the complementary nucleotides combine with natural sequences produced by the cell to form duplexes and to block either transcription or translation. The designation “negative” can refer to the antisense strand, and the designation “positive” can refer to the sense strand.

As used herein, the term “biologically active,” refers to a protein having structural, regulatory, or biochemical functions of a naturally occurring molecule. Likewise, “immunologically active” refers to the capability of the natural, recombinant, or synthetic I-2, or of any oligopeptide thereof, to induce a specific immune response in appropriate animals or Gells and to bind with specific antibodies.

The terms “complementary” or “complementarity,” as used herein, refer to the natural binding of polynucleotides under permissive salt and temperature conditions by base pairing. For example, the sequence “A-G-T” binds to the complementary sequence “T-C-A.” Complementarity between two single-stranded molecules can be “partial,” such that only some of the nucleic acids bind, or it can be “complete,” such that total complementarity exists between the single stranded molecules. The degree of complementarity between nucleic acid strands has significant effects on the efficiency and strength of the hybridization between the nucleic acid strands. This is of particular importance in amplification reactions, which depend upon binding between nucleic acids strands, and in the design and use of peptide nucleic acid (PNA) molecules.

A “composition comprising a given polynucleotide sequence” or a “composition comprising a given amino acid sequence,” as these terms are used herein, refer broadly to any composition containing the given polynucleotide or amino acid sequence. The composition can comprise a dry formulation, an aqueous solution, or a sterile composition. Compositions comprising polynucleotide sequences encoding I-2 or fragments of I-2 can be employed as hybridization probes. The probes can be stored in freeze-dried form and can be associated with a stabilizing agent such as a carbohydrate. In hybridizations, the probe can be deployed in an aqueous solution containing salts (e.g., NaCl), detergents (e.g., SDS), and other components (e.g., Denhardt's solution, dry milk, salmon sperm DNA, etc.).

“Consensus sequence,” as used herein, refers to a nucleic acid sequence which has been resequenced to resolve uncalled bases, extended using XL-PCRTM (Perkin Elmer, Norwalk, Conn.) in the 5′ and/or the 3′direction, and resequenced, or which has been assembled from the overlapping sequences of more than one Incyte Clone using a computer program for fragment assembly, such as the GELVIEW.TM. Fragment Assembly system (GCG, Madison, Wis.). Some sequences have been both extended and assembled to produce the consensus sequence.

As used herein, the term “correlates with expression of a polynucleotide” indicates that the detection of the presence of nucleic acids, the same or related to a nucleic acid sequence encoding I-2, by northern analysis is indicative of the presence of nucleic acids encoding I-2 in a sample, and thereby correlates with expression of the transcript from the polynucleotide encoding 1-2.

A “deletion,” as the term is used herein, refers to a change in the amino acid or nucleotide sequence that results in the absence of one or more amino acid residues or nucleotides.

The term “derivative,” as used herein, refers to the chemical modification of I-2, of a polynucleotide sequence encoding I-2, or of a polynucleotide sequence complementary to a polynucleotide sequence encoding I-2. Chemical modifications of a polynucleotide sequence can include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

The term “homology,” as used herein, refers to a degree of complementarity. There can be partial homology or complete homology. The word “identity” can substitute for the word “homology.” A partially complementary sequence that at least partially inhibits an identical sequence from hybridizing to a target nucleic acid is referred to as “substantially homologous.” The inhibition of hybridization of the completely complementary sequence to the target sequence can be examined using a hybridization assay (Southern or northern blot, solution hybridization, and the like) under conditions of reduced stringency. A substantially homologous sequence or hybridization probe can compete for and inhibit the binding of a completely homologous sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding can be tested by the use of a second target sequence which lacks even a partial degree of complementarity (e.g., less than about 30% homology or identity). In the absence of non-specific binding, the substantially homologous sequence or probe can not hybridize to the second non-complementary target sequence.

The phrases “percent identity” and “% identity” refer to the percentage of sequence similarity found in a comparison of two or more amino acid or nucleic acid sequences. Percent identity can be determined electronically, e.g., by using the MegAlign.TM. program (DNASTAR, Inc., Madison Wis.). The MegAlign.TM. program can create alignments between two or more sequences according to different methods, e.g., the clustal method. (See, e.g., Higgins, D. G. and P. M. Sharp (1988) Gene 73:237-244.) The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent identity between nucleic acid sequences can also be counted or calculated by other methods known in the art, e.g., the Jotun Hein method. (See, e.g., Hein, J. (1990) Methods Enzymol. 183:626-645.) Identity between sequences can also be determined by other methods known in the art, e.g., by varying hybridization conditions.

“Hybridization,” as the term is used herein, refers to any process by which a strand of nucleic acid binds with a complementary strand through base pairing.

The words “insertion” or “addition,” as used herein, refer to changes in an amino acid or nucleotide sequence resulting in the addition of one or more amino acid residues or nucleotides, respectively, to the sequence found in the naturally occurring molecule.

“Immune response” can refer to conditions associated with inflammation, trauma, immune disorders, or infectious or genetic disease, etc. These conditions can be characterized by expression of various factors, e.g., cytokines, chemokines, and other signaling molecules, which can affect cellular and systemic defense systems.

The term “microarray,” as used herein, refers to an arrangement of distinct polynucleotides arrayed on a substrate, e.g., paper, nylon or any other type of membrane, filter, chip, glass slide, or any other suitable solid support.

The terms “element” or “array element” as used herein in a microarray context, refer to hybridizable polynucleotides arranged on the surface of a substrate.

The term “modulate,” as it appears herein, refers to a change in the activity of I-2. For example, modulation can cause an increase or a decrease in protein activity, binding characteristics, or any other biological, functional, or immunological properties of 1-2.

The phrases “nucleic acid” or “nucleic acid sequence,” as used herein, refer to an oligonucleotide, nucleotide, polynucleotide, or any fragment thereof, to DNA or RNA of genomic or synthetic origin which can be single-stranded or double-stranded and can represent the sense or the antisense strand, to peptide nucleic acid (PNA), or to any DNA-like or RNA-like material. In this context, “fragments” refers to those nucleic acid sequences which are greater than about 60 nucleotides in length, and most preferably are at least about 100 nucleotides, at least about 1000 nucleotides, or at least about 10,000 nucleotides in length.

The terms “operably associated” and “operably linked,” as used herein, refer to functionally related nucleic acid sequences. A promoter is operably associated or operably linked with a coding sequence if the promoter controls the transcription of the encoded polypeptide. While operably associated or operably linked nucleic acid sequences can be contiguous and in the same reading frame, certain genetic elements, e.g., repressor genes, are not contiguously linked to the sequence encoding the polypeptide but still bind to operator sequences that control expression of the polypeptide.

The term “oligonucleotide,” as used herein, refers to a nucleic acid sequence of at least about 6 nucleotides to 60 nucleotides, preferably about 15 to 30 nucleotides, and most preferably about 20 to 25 nucleotides, which can be used in PCR amplification or in a hybridization assay or microarray. As used herein, the term “oligonucleotide” is substantially equivalent to the terms “amplimer,” “primer,” “oligomer,” and “probe,” as these terms are commonly defined in the art.

“Peptide nucleic acid” (PNA), as used herein, refers to an antisense molecule or to anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA and RNA and stop transcript elongation, and can be pegylated to extend their lifespan in the cell. (See, e.g., Nielsen, P. E. et al. (1993) Anticancer Drug Des. 8:53-63.)

The term “sample,” as used herein, is used in its broadest sense. A biological sample suspected of containing nucleic acids encoding I-2, or fragments thereof, or I-2 itself, can comprise a bodily fluid; an extract from a cell, chromosome, organelle, or membrane isolated from a cell; a cell; genomic DNA, RNA, or cDNA, in solution or bound to a solid support; a tissue; a tissue print; etc.

As used herein, the terms “specific binding” and “specifically binding” refer to that interaction between a protein or peptide and an agonist, an antibody, or an antagonist. The interaction is dependent upon the presence of a particular structure of the protein, e.g., the antigenic determinant or epitope, recognized by the binding molecule. For example, if an antibody is specific for epitope “A,” the presence of a polypeptide containing the epitope A, or the presence of free unlabeled A, in a reaction containing free labeled A and the antibody can reduce the amount of labeled A that binds to the antibody.

As used herein, the term “stringent conditions” refers to conditions which permit hybridization between polynucleotide sequences and the claimed polynucleotide sequences. Suitably stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature.

For example, hybridization under high stringency conditions could occur in about 50% formamide at about 37° C. to 42° C. Hybridization could occur under reduced stringency conditions in about 35% to 25% formamide at about 30° C. to 35° C. In particular, hybridization could occur under high stringency conditions at 42° C. in 50% formamide, 5×SSPE, 0.3% SDS, and 200 mg/ml sheared and denatured salmon sperm DNA. Hybridization could occur under reduced stringency conditions as described above, but in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art.

The term “substantially purified,” as used herein, refers to nucleic acid or amino acid sequences that are removed from their natural environment and are isolated or separated, and are at least about 60% free, preferably about 75% free, and most preferably about 90% free from other components with which they are naturally associated.

A “substitution,” as used herein, refers to the replacement of one or more amino acids or nucleotides by different amino acids or nucleotides, respectively.

“Transformation,” as defined herein, describes a process by which exogenous DNA enters and changes a recipient cell. Transformation can occur under natural or artificial conditions according to various methods well known in the art, and can rely on any known method for the insertion of foreign nucleic acid sequences into a prokaryotic or eukaryotic host cell. The method for transformation is selected based on the type of host cell being transformed and can include, but is not limited to, viral infection, electroporation, heat shock, lipofection, and particle bombardment. The term “transformed” cells includes stably transformed cells in which the inserted DNA is capable of replication either as an autonomously replicating plasmid or as part of the host chromosome, as well as transiently transformed cells which express the inserted DNA or —RNA for limited periods of time.

A “variant” of I-2, as used herein, refers to an amino acid sequence that is altered by one or more amino acids. The variant can have “conservative” changes, wherein a substituted amino acid has similar structural or chemical properties (e.g., replacement of leucine with isoleucine). More rarely, a variant can have “nonconservative” changes (e.g., replacement of glycine with tryptophan). Analogous minor variations can also include amino acid deletions or insertions, or both. Guidance in determining which amino acid residues can be substituted, inserted, or deleted without abolishing biological or immunological activity can be found using computer programs well known in the art, for example, LASERGENE™ software.

As used herein, the term “therapeutically effective amount” of an agent shall refer to an amount of that agent which is physiologically significant and improves an individual's health. An agent is “physiologically significant”if its presence results in a change in the physiology of the recipient human. For example, in the treatment of a pathological condition, administration of an agent which relieves or arrests further progress of the condition would be considered both physiologically significant and therapeutically effective.

Protein phosphatase activity is increased in the failing heart, a finding that has major impact on the state of phosphorylation of key sarcoplasmic reticulum proteins and consequently adverse impact on cardiac muscle contractility and relaxation. Selective inhibiting of protein phosphatase activity in the heart can have a direct impact on improving cardiac muscle contraction. Inhibitor-2 (I-2) is a potent inhibitior of type-1 protein phosphatase (PP1) activity and it has yet not been cloned from cardiac tissue. Using the reverse and forward primers and RT-PCR technique, a full-length cDNA encoding I-2 was synthesized from RNA isolated from LV myocardium of normal (NL) dog. The resulting PCR product of Mr 0.662 kb was subsequently cloned into pCRT7 TOPO vector and then transformed in cloning E. coli. The identity and orientation of the cloned product was examined by restriction enzyme and sequencing. The plasmid containing the right-orientation of the gene was then transformed in an E. coli strain capable of expressing the gene. The gene was induced by isopropyl β-D-thiogalactoside (IPTG) at different time points varying from 0 hours to 4 hours. At four hours, maximal expression of I-2 protein was observed in the presence of IPTG, whereas negligible amount of this protein was induced in the absence of IPTG. The identity of I-2 protein was confirmed by immunoblotting using I-2 antibody and by its ability to inhibit PP1 activity. The I-2 protein was approximately ? fold higher in the cytosol than membrane of gene expressing E. coli. The sequence of dog LV I-2 cDNA was found to have approximately 99% homology with that of human colon and lymphocyte, 91% with rat brain, and 86% with rabbit skeletal muscle. The only difference in the predicted amino acid sequence of dog LV I-2 was alanine at position 73 that was substituted for threonine in human colon I-2. A full length cDNA sequence of I-2 from human LV myocardium was also cloned.

In a failing heart, it has been found that protein phosphatase activity, particularly of type-1, was increased an abnormality that can account for the reduced left ventricular (LV) that is characteristic of heart failure (HF) state. Thus, selective inhibition of type-1 protein phosphatase (PP1) activity in the heart muscle leads to improvement of global LV function. At present, a synthetic inhibitor that is specific to PP1 activity is not available. In skeletal muscle extract, two heat and acid stable low molecular weight proteins of approximately 26-32 kDa have been reported to be present (Cohen et al., 1998). These proteins have been named in inhibitor-1 and inhibitor-2 (I-2). Both inhibitors inhibit PP1 activity. However, inhibitor-1 inhibits phosphatase activity only when it has been phosphorylated by cAMP-dependent protein kinase, whereas I-2 inhibits phosphatase activity regardless of its phosphorylation state (Cohen et al., 1998). Though the presence of I-2 has been reported in rabbit heart (Roach et al., 1985), its presence in dog and human hearts has not yet been documented. Results from the laboratory indicate that I-2 is present in dog and human hearts. The gene encoding I-2 has been cloned from rat brain (6Sakagami, et al., 1995), rabbit skeletal muscle and liver (Zhang et al., 1994; Park et al., 1994) and human colon (Sanseau et al., 1994), whereas this gene has not been cloned from the heart of any species. The efforts have focused on isolating and cloning a full-length cDNA capable of encoding I-2 from dog heart and human heart. The dog gene can be used for efficacy and safety testing in dogs with heart failure (HF). The ultimate objective is to use I-2 gene therapy for the treatment of patients with chronic HF. The therapy can be based on overexpression of I-2 in the heart fro the intended purpose of inhibiting PP1 activity.

The present invention is directed to methods for administering I-2, or a compound structurally related to I-2, to an individual to diminish myocardial infarction and delay cell injury or death in ischemic cardiac tissue. It is contemplated that beneficial therapeutic effects is achieved if I-2, or a compound structurally related to I-2, is administered either before or after the onset of a myocardial infarction. For therapeutic applications, a person having ordinary skill in the art of molecular pharmacology would be able to determine, without undue experimentation, the appropriate dosages and routes of administration of the novel pharmacological compounds of the present invention.

I-2 or a fragment or derivative thereof can also be administered to a subject to treat or prevent an immune disorder. Such disorders can include, but are not limited to, AIDS, Addison's disease, adult respiratory distress syndrome, allergies, ankylosing spondylitis, amyloidosis, anemia, asthma, atherosclerosis, autoimmune hemolytic anemia, autoimmune thyroiditis, bronchitis, cholecystitis, contact dermatitis, Crohn's disease, atopic dermatitis, dermatomyositis, diabetes mellitus, emphysema, erythema nodosum, atrophic gastritis, glomerulonephritis, Goodpasture's syndrome, gout, Graves' disease, Hashimoto's thyroiditis, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, systemic anaphylaxis, systemic lupus erythematosus, systemic sclerosis, ulcerative colitis, Werner syndrome, and complications of cancer, hemodialysis, and extracorporeal circulation, viral, bacterial, fungal, parasitic, protozoal, and helminthic infections, and trauma.

In another embodiment, a vector capable of expressing I-2 or a fragment or derivative thereof can be administered to a subject to treat or prevent an immune disorder including, but not limited to, those described above. [Is this applicable?]

In a further embodiment, a pharmaceutical composition comprising a substantially purified I-2 in conjunction with a suitable pharmaceutical carrier can be administered to a subject to treat or prevent an immune disorder including, but not limited to, those provided above. [Is this applicable?]

In another embodiment, I-2 or a fragment or derivative thereof can be administered to a subject to treat or prevent a reproductive disorder. Such disorders can include, but are not limited to, disorders of prolactin production; infertility, including tubal disease, ovulatory defects, and endometriosis; disruptions of the estrous cycle, disruptions of the menstrual cycle, polycystic ovary syndrome, ovarian hyperstimulation syndrome, endometrial and ovarian tumors, autoimmune disorders, ectopic pregnancy, and teratogenesis; cancer of the breast, uterine fibroids, fibrocystic breast disease, and galactorrhea; disruptions of spermatogenesis, abnormal sperm physiology, cancer of the testis, cancer of the prostate, benign prostatic hyperplasia, prostatitis, Peyronie's disease, carcinoma of the male breast, and gynecomastia.

In another embodiment, a vector capable of expressing I-2 or a fragment or derivative thereof can be administered to a subject to treat or prevent a reproductive disorder including, but not limited to, those described above. [Is this applicable?]

In a further embodiment, a pharmaceutical composition comprising a substantially purified I-2 in conjunction with a suitable pharmaceutical carrier can be administered to a subject to treat or prevent a reproductive disorder including, but not limited to, those provided above. [Is this applicable?]

In other embodiments, any of the proteins, antagonists, antibodies, agonists, complementary sequences, or vectors of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one can be able to achieve therapeutic efficacy with lower dosages of each agent, thus reducing the potential for adverse side effects.

An antagonist of I-2 can be produced using methods which are generally known in the art. In particular, purified I-2 can be used to produce antibodies or to screen libraries of pharmaceutical agents to identify those which specifically bind I-2. Antibodies to I-2 can also be generated using methods that are well known in the art. Such antibodies can include, but are not limited to, polyclonal, monoclonal, chimeric, and single chain antibodies, Fab fragments, and fragments produced by a Fab expression library. Neutralizing antibodies (i.e., those which inhibit dimer formation) are especially preferred for therapeutic use.

For the production of antibodies, various hosts including goats, rabbits, rats, mice, humans, and others can be immunized by injection with I-2 or with any fragment or oligopeptide thereof which has immunogenic properties. Depending on the host species, various adjuvants can be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to I-2 have an amino acid sequence consisting of at least about five amino acids, and, more preferably, of at least about 10 amino acids. It is also preferable that these oligopeptides, peptides, or fragments are identical to a portion of the amino acid sequence of the natural protein and contain the entire amino acid sequence of a small, naturally occurring molecule. Short stretches of I-2 amino acids can be fused with those of another protein, such as KLH, and antibodies to the chimeric molecule can be produced.

Monoclonal antibodies to I-2 can be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. 81:6851-6855; Neuberger, M. S. et al. (1984) Nature 312:604-608; and Takeda, S. et al. (1985) Nature 314:452-454.) Alternatively, techniques described for the production of single chain antibodies can be adapted, using methods known in the art, to produce I-2-specific single chain antibodies. Antibodies with related specificity, but of distinct idiotypic composition, can be generated by chain shuffling from random combinatorial immunoglobulin libraries. (See, e.g., Burton D. R. (1991) Pros. Natl. Acad. Sci. 88:10134-10137.)

Antibodies can also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. 86: 3833-3837; and Winter, G. et al. (1991) Nature 349:293-299.)

Antibody fragments which contain specific binding sites for I-2 can also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries can be constructed to allow rapid and easy identification of monoclonal Fab fragments with the desired specificity. (See, e.g., Huse, W. D. et al. (1989) Science 246:1275-1281.)

Various immunoassays can be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between I-2 and its specific antibody. A two-site, monoclonal-based immunoassay utilizing monoclonal antibodies reactive to two non-interfering I-2 epitopes is preferred, but a competitive binding assay can also be employed. (Maddox, supra.)

In another embodiment of the invention, the polynucleotides encoding I-2, or any fragment or complement thereof, can be used for therapeutic purposes. In one aspect, the complement of the polynucleotide encoding I-2 can be used in situations in which it would be desirable to block the transcription of the mRNA. In particular, cells can be transformed with sequences complementary to polynucleotides encoding I-2. Thus, complementary molecules or fragments can be used to modulate protein phosphatase activity, or to achieve regulation of gene function. Such technology is now well known in the art, and sense or antisense oligonucleotides or larger fragments can be designed from various locations along the coding or control regions of sequences encoding 1-2.

Expression vectors derived from retroviruses, adenoviruses, or herpes or vaccinia viruses, or from various bacterial plasmids, can be used for delivery of nucleotide sequences to the targeted organ, tissue, or cell population. Methods which are well known to those skilled in the art can be used to construct vectors which can express nucleic acid sequences complementary to the polynucleotides of the gene encoding PPRM. (See, e.g., Sambrook, supra; and Ausubel, supra.)

Genes encoding I-2 can be turned off by transforming a cell or tissue with expression vectors which express high levels of a polynucleotide, or fragment thereof, encoding I-2. Such constructs can be used to introduce untranslatable sense or antisense sequences into a cell. Even in the absence of integration into the DNA, such vectors can continue to transcribe RNA molecules until they are disabled by endogenous nucleases. Transient expression can last for a month or more with a non-replicating vector, and can last even longer if appropriate replication elements are part of the vector system.

As mentioned above, modifications of gene expression can be obtained by designing complementary sequences or antisense molecules (DNA, RNA, or PNA) to the control, 5′, or regulatory regions of the gene encoding I-2. Oligonucleotides derived from the transcription initiation site, e.g., between about positions −10 and +10 from the start site, are preferred. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing Co., Mt. Kisco, N.Y., pp. 163-177.) A complementary sequence or antisense molecule can also be designed to block translation of mRNA by preventing the transcript from binding to ribosomes.

Ribozymes, enzymatic RNA molecules, can also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules can specifically and efficiently catalyze endonucleolytic cleavage of sequences encoding 1-2.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, can be evaluated for secondary structural features which can render the oligonucleotide inoperable. The suitability of candidate targets can also be evaluated by testing accessibility to hybridization with complementary oligonucleotides using ribonuclease protection assays.

Complementary ribonucleic acid molecules and ribozymes of the invention can be prepared by any method known in the art for the synthesis of nucleic acid molecules. These include techniques for chemically synthesizing oligonucleotides such as solid phase phosphoramidite chemical synthesis. Alternatively, RNA molecules can be generated by in vitro and in vivo transcription of DNA sequences encoding I-2. Such DNA sequences can be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules can be modified to increase intracellular stability and half-life. Possible modifications include, but are not limited to, the addition of flanking sequences at the 5′ and/or 3′ ends of the molecule, or the use of phosphorothioate or 2′O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine,-cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors can be introduced into stem cells taken from the patient and clonally propagated for autologous transplant back into that same patient. Delivery by transfection, by liposome injections, or by polycationic amino polymers can be achieved using methods which are well known in the art. (See, e.g., Goldman, C. K. et al. (1997) Nature Biotechnology 15:462-466.)

Any of the therapeutic methods described above can be applied to any subject in need of such therapy, including, for example, mammals such as dogs, cats, cows, horses, rabbits, monkeys, and most preferably, humans.

An additional embodiment of the invention relates to the administration of a pharmaceutical or sterile composition, in conjunction with a pharmaceutically acceptable carrier, for any of the therapeutic effects discussed above. Such pharmaceutical compositions can consist of I-2, antibodies to I-2, and mimetics, agonists, antagonists, or inhibitors of I-2. The compositions can be administered alone or in combination with at least one other agent, such as a stabilizing compound, which can be administered in any sterile, biocompatible pharmaceutical carrier including, but not limited to, saline, buffered saline, dextrose, and water. The compositions can be administered to a patient alone, or in combination with other agents, drugs, or hormones.

The pharmaceutical compositions utilized in this invention can be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions can contain suitable pharmaceutically-acceptable carriers comprising excipients and auxiliaries which facilitate processing of the active compounds into preparations which can be used pharmaceutically. Further details on techniques for formulation and administration can be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents can be added, such as the cross-linked polyvinyl pyrrolidone, agar, and alginic acid or a salt thereof, such as sodium alginate.

Dragee cores can be used in conjunction with suitable coatings, such as concentrated sugar solutions, which can also contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks's solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers can also be used for delivery. Optionally, the suspension can also contain suitable stabilizers or agents to increase the solubility of the compounds and allow for the preparation of highly concentrated solutions.

For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes.

The pharmaceutical composition can be provided as a salt and can be formed with many acids, including but not limited to, hydrochloric, sulfuric, acetic, lactic, tartaric, malic, and succinic acid. Salts tend to be more soluble in aqueous or other protonic solvents than are the corresponding free base forms. In other cases, the preferred preparation can be a lyophilized powder which can contain any or all of the following: 1 mM to 50 mM histidine, 0.1% to 2% sucrose, and 2% to 7% mannitol, at a pH range of 4.5 to 5.5, that is combined with buffer prior to use.

After pharmaceutical compositions have been prepared, they can be placed in an appropriate container and labeled for treatment of an indicated condition. For administration of I-2, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells or in animal models such as mice, rats, rabbits, dogs, or pigs. An animal model can also be used to determine the appropriate concentration range and route of administration. Such information can then be used to determine useful doses and routes for administration in humans.

A therapeutically effective dose refers to that amount of active ingredient, for example I-2 or fragments thereof, antibodies of I-2, and agonists, antagonists or inhibitors of I-2, which ameliorates the symptoms or condition. Therapeutic efficacy and toxicity can be determined by standard pharmaceutical procedures in cell cultures or with experimental animals, such as by calculating the ED₅₀(the dose therapeutically effective in 50% of the population) or LD₅₀(the dose lethal to 50% of the population) statistics. The dose ratio of therapeutic to toxic effects is the therapeutic index, and it can be expressed as the ED₅₀/LD₅₀ ratio. Pharmaceutical compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage can be determined by the practitioner, in light of factors related to the subject requiring treatment. Dosage and administration are adjusted to provide sufficient levels of the active moiety or to maintain the desired effect. Factors which can be taken into account include the severity of the disease state, the general health of the subject, the age, weight, and gender of the subject, time and frequency of administration, drug combination(s), reaction sensitivities, and response to therapy. Long-acting pharmaceutical compositions can be administered every 3 to 4 days, every week, or biweekly depending on the half-life and clearance rate of the particular formulation.

Normal dosage amounts can vary from about 0.1 mg to 100,000 mg, up to a total dose of about 1 gram, depending upon the route of administration. Guidance as to particular dosages and methods of delivery is provided in the literature and generally available to practitioners in the art. Those skilled in the art can employ different formulations for nucleotides than for proteins or their inhibitors. Similarly, delivery of polynucleotides or polypeptides can be specific to particular cells, conditions, locations, etc.

In another embodiment, one can use competitive drug screening assays in which neutralizing antibodies capable of inhibiting PP1 specifically compete with a test compound for inhibiting PP1. In this manner, antibodies can be used to detect the presence of any peptide which shares one or more antigenic determinants with I-2.

In additional embodiments, the nucleotide sequences which encode I-2 can be used in any molecular biology techniques that have yet to be developed, provided the new techniques rely on properties of nucleotide sequences that are currently known, including, but not limited to, such properties as the triplet genetic code and specific base pair interactions.

### EXAMPLES

Methods

General methods in molecular biology: Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences (Testoni et al, 1996, Blood 87:3822.)

General methods in immunology: Standard methods in immunology known in the art and not specifically described are generally followed as in Stites et al.(eds), Basic and Clinical Immunology (8th Edition), Appleton & Lange, Norwalk, Conn. (1994) and Mishell and Shiigi (eds), Selected Methods in Cellular Immunology, W. H. Freeman and Co., New York (1980).

For Gene Therapy:

By gene therapy as used herein refers to the transfer of genetic material (e.g DNA or RNA) of interest into a host to treat or prevent a genetic or acquired disease or condition phenotype. The genetic material of interest encodes a product (e.g. a protein, polypeptide, peptide, functional RNA, antisense) whose production in vivo is desired. For example, the genetic material of interest can encode a hormone, receptor, enzyme, polypeptide or peptide of therapeutic value. Alternatively, the genetic material of interest encodes a suicide gene. For a review see, in general, the text “Gene Therapy” (Advances in Pharmacology 40, Academic Press, 1997).

Two basic approaches to gene therapy have evolved: (1) ex vivo and (2) in vivo gene therapy. In ex vivo gene therapy cells are removed from a patient, and while being cultured are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to express the transfected genetic material in situ.

In in vivo gene therapy, target cells are not removed from the subject rather the genetic material to be transferred is introduced into the cells of the recipient organism in situ, that is within the recipient. In an alternative embodiment, if the host gene is defective, the gene is repaired in situ [Culver, 1998]. These genetically altered cells have been shown to express the transfected genetic material in situ.

The gene expression vehicle is capable of delivery/transfer of heterologous nucleic acid into a host cell. The expression vehicle can include elements to control targeting, expression and transcription of the nrucleic acid in a cell selective manner as is known in the art. It should be noted that often the 5′UTR and/or 3′UTR of the gene can be replaced by the 5′UTR and/or 3′UTR of the expression vehicle. Therefore as used herein the expression vehicle can, as needed, not include the 5′UTR and/or 3′UTR of the actual gene to be transferred and only include the specific amino acid coding region.

The expression vehicle can include a promotor for controlling transcription of the heterologous material and can be either a constitutive or inducible promotor to allow selective transcription. Enhancers that can be required to obtain necessary transcription levels can optionally be included. Enhancers are generally any non-translated DNA sequence which works contiguously with the coding sequence (in cis) to change the basal transcription level dictated by the promoter. The expression vehicle can also include a selection gene as described herein below.

Vectors can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Anr Arbor, Mich. (1995), Vectors: A Survey of Molecular Cloning Vectors and Their Uses, Butterworths, Boston Mass. (1988) and Gilboa et al (1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector for introducing and expressing recombinant sequences is the adenovirus derived vector Adenop53 TK. This vector expresses a herpes virus thymidine kinase (TK) gene for either positive or negative selection and an expression cassette for desired recombinant sequences. This vector can be used to infect cells that have an adenovirus receptor which includes most cancers of epithelial origin as well as others. This vector as well as others that exhibit similar desired functions can be used to treat a mixed population of cells and can include, for example, an in vitro or ex vivo culture of cells, a tissue or a human subject.

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation can not occur.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods of the invention can depend on desired cell type to be targeted and can be known to those skilled in the art. For example, if breast cancer is to be treated then a vector specific for such epithelial cells would be used. Likewise, if diseases or pathological conditions of the hematopoietic system are to be treated, then a viral vector that is specific for blood cells and their precursors, preferably for the specific type of hematopoietic cell, would be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed can not contain a genome and therefore cannot proceed through subsequent rounds of infection. The specific type of vector can depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment, administration can also be performed by, for example, intravenous or subcutaneous injection into the subject. Injection of the viral vectors into a spinal fluid can also be used as a mode of administration, especially in the case of neuro-degenerative diseases. Following injection, the viral vectors can circulate until they recognize host cells with the appropriate target specificity for infection.

An alternate mode of administration can be by direct inoculation locally at the site of the disease or pathological condition or by inoculation into the vascular system supplying the site with nutrients or into the spinal fluid. Local administration is advantageous because there is no dilution effect and, therefore, a smaller dose is required to achieve expression in a majority of the targeted cells. Additionally, local inoculation can alleviate the targeting requirement required with other forms of administration since a vector can be used that infects all cells in the inoculated area. If expression is desired in only a specific subset of cells within the inoculated area, then promoter and regulatory elements that are specific for the desired subset can be used to accomplish this goal. Such non-targeting vectors can be, for example, viral vectors, viral genome, plasmids, phagemids and the like. Transfection vehicles such as liposomes can also be used to introduce the non-viral vectors described above into recipient cells within the inoculated area. Such transfection vehicles are known by one skilled within the art.

Delivery of Gene Products/Therapeutics (Compound):

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound or as pharmaceutically acceptable salt and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the mice or other experimental animals exemplified herein which treatment has a length proportional to the length of the disease process and drug effectiveness. The doses can be single doses or multiple doses over a period of several days, but single doses are preferred.

The doses can be single doses or multiple doses over a period of several days. The treatment generally has a length proportional to the length of the disease process and drug effectiveness and the patient species being treated.

When administering the compound of the present invention parenterally, it can generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it can be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques which deliver it orally or intravenously and retain the biological activity are preferred.

In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used. The quantity to be administered can vary for the patient being treated and can vary from about 100 ng/kg of body weight to 100 mg/kg of body weight per day and preferably can be from 10 mg/kg to 10 mg/kg per day.

### Example 1

METHODS: Cloning of-I-2: Total cellular RNA was isolated from frozen LV myocardial tissue of normal dogs using a RNA Stat-60 kit and the instructions obtained form Tel-Test “B”, Inc. (Firewoods, Tex.). The concentration of the isolated RNA was quantitated spectrophotometrically by measuring absorbance at 260 nm/280 nm. If this ratio is between 1.7-2.0, the isolated RNA is considered of good quality. Approximately 8 μg of the isolated RNA was reverse-transcribed into cDNA in a total assay volume of 80 μl consisting of 3.6 mM each of dNTP (dATP, dTTP, dGTP, and dCTP), 40 units recombinant Rnasin (Rnase inhibitor), 6 μM oligo (dT) primer, and 1 unit MMLV reverse transcriptase, 10 mM Tris-HCL, pH8.3, 75 mM KCl, 10 mM DTT, and 3 mM MgCl₂. The assay tube was incubated at 42° C. for 60 minutes and then reaction was immediately terminated at 95° C. for 10 minutes. For PCR, 10 μl of the first-strand cDNA was added into 90 μl of a reaction consisting of 20 pmol forward and reverse primers specific for I-2, 200 μM of each dNTP, 10 mM Tris-HCl, pH 8.8, 50 mM KCl, 0.1% Triton-X100, 1 unit Taq DNA polymerase (Promega, Madison, Wis.) and 3 mM MgCl₂. The assay tubes were kept in PCR machine (Mastercycler Gradient, Eppendorf) and that was programmed for one cycle (94° C., 3′2″)/40 cycles (denaturation: 94° C., 1′; annealing: 52° C., 1′; extension 72° C., 1′2″; delay 1″) and then finally hold at 4° C. The forward (5′CCAATGGCGGGCCTCGACGGCCTC-3′) and reverse (5′TCGTAATTTGTTTGCTGTTGGTCACT-3;) primers for dog heart I-2 were designed from the published human colon I-2(Sanseau et al., 1994).

The PCR product was analyzed by electrophoresing 10 μl of the reaction mixture on 2% agarose gel electrophoresis followed by ethidium bromide staining. A single band of approximately 0.612 kb was excised from the agarose gel and purified using a Qiagen purification kit. The purity of the purified DNA was checked by agarose gel electrophoresis. The purified cDNA was ligated to PCR T7 TOPO and then transformed into competent TOP10F′ cells, and plated on Liquid Broth-ampicillin-agar plates according to the instructions of a kit supplied by manufacturing (Invitrogen). After incubation overnight at 37° C., about four colonies were picked up, plasmid from each colony was isolated, and the presence of I-2 gene in right orientation was confirmed by using the restriction enzyme, Hind III and sequencing. Sequencing of the plasmid was carried out by a DNA Sequencing Facility (Wayne State University, Detroit, Mich.). The colony having right orientation of full-length I-2 gene was stored in glycerol solution at −70° C.

Expression of Inhibitor-2 Gene: Approximately 10 ng plasmid isolated from the recombinant TOP10F′ cells (used in cloning) was transformed into competent bacteria BL21 (DE3) plyS cells (gene expression bacteria) by using a kit and instructions from the supplier (Invitrogen). Transformed bacterial cells were grown in 10 ml LB medium containing 100 μg/ml ampicillin and 34 μg/ml chloramphenicol at 37° C. for 2 hours. Subsequently, the medium was divided into two equally parts, each consisting of 5 ml. In one part, 1 mM isopropyl β-D-thiogalactoside (IPTG) was added and the second part received no IPTG. At different time points with 1 hour apart, approximately 500 μl bacterial broth was taken out up to 4 hours of incubation and then immediately centrifuged at 12,000 g for 10 minutes and the pellet was frozen at −70° C. The pellet was homogenized as previously described (Gupta et al., 1997) in 50 mM Tris-HCL, pH 7.4, 0.3 M sucrose, and protease inhibitors (20.8 mM benzamidine, 0.8 mg/ml of aprotinin and leupeptin, and 0.4 μg/l antipain). Protein was assayed using the Bradford approach with bovine serum albumin being used as a standard (Bradford et al., 1976).

Effect of the Recombinant I-2 on PP1 Activity: Using ³²P-labeled phosphorylase a as the substrate, the assay for PP1 activity was set up in a total assay volume of 50 μl consisting of 50 μg ³²P-labelled phosphorylase a (approximately 600 dpm/pmol), 50 mM Tris-HCl, pH 7.4, 0.25 mM EGTA, 10 mM â-mercaptoethanol, 5 mM caffeine, and the purified catalytic subunit of PP1 from rabbit skeletal muscle (enough to hydrolyze about 30% of the substrate). The homogenate of bacterial extract was incubated in a boiling water bath for 10′ and then immediately cooled down. Approximately, 10 μg of the heat treated bacterial extract was preincubated with the catalytic subunit of PP1 at 34° C. for 1 minute and then the assay was initiated by adding mixture containing everything as described above except the phosphatase. After incubation at 35° C. for 10 minutes, the reaction was terminated and ⁼P released was counted as previously described (Gupta et al., 1996). The phosphatase activity was expressed as % PP1 activity.

Immunoblotting: To determine the expression level of I-2 in gene-expressing bacterial cells, frozen cells were lysed in 2% SDS and then boiled for 5 minutes. After cooling at room temperature, the mixture was centrifuged at 12,000 g for 10′ and the clear supernatant was saved for analysis. Approximately 5-20 μg protein was electrophoresed on 12% SDS-PAGE and then separated proteins were electrophoretically transferred to a nitrocellulose membrane as previously described (Gupta et al., 1997). The accuracy of the electrotransfer of each sample was confirmed by staining the membranes with 0.1% amido black. For the immunoreaction, the membrane was incubated with 500× diluted monoclonal antibody of I-2(Transduction) and then the membrane was incubated with a secondary antibody as previously described (Gupta et al., 1997; Gupta et al., 1999). The antibody-binding protein was visualized by autoradiography following incubating the membrane with ECL-color developing reagents (Amersham). In all circumstances, it is ensured that the antibody was present in excess amount over the antigen.

Results

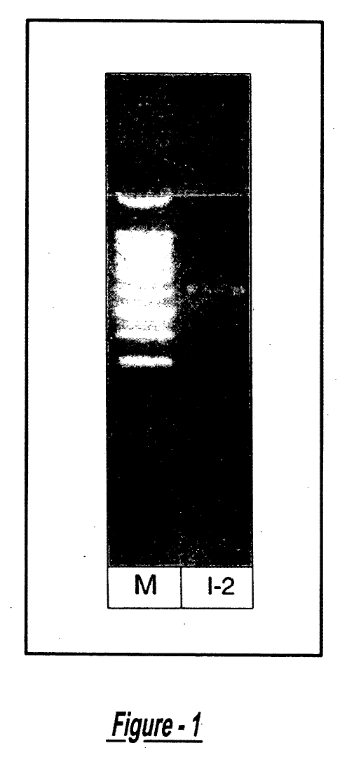

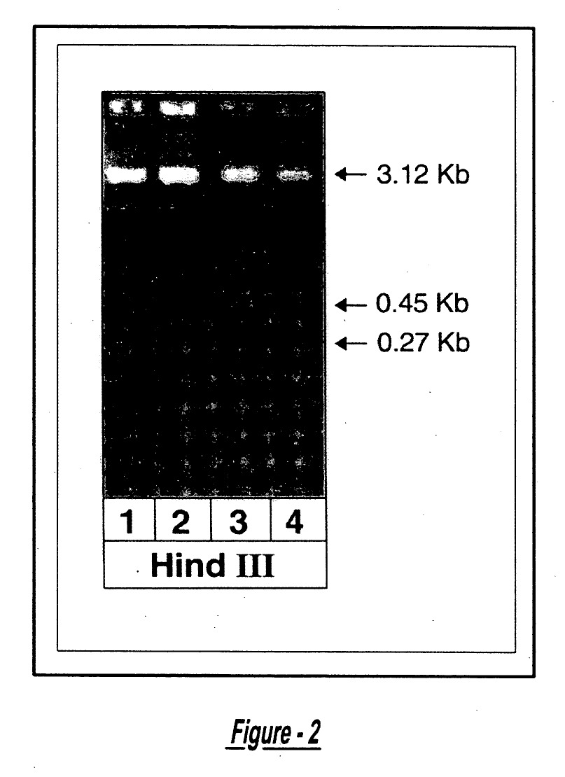

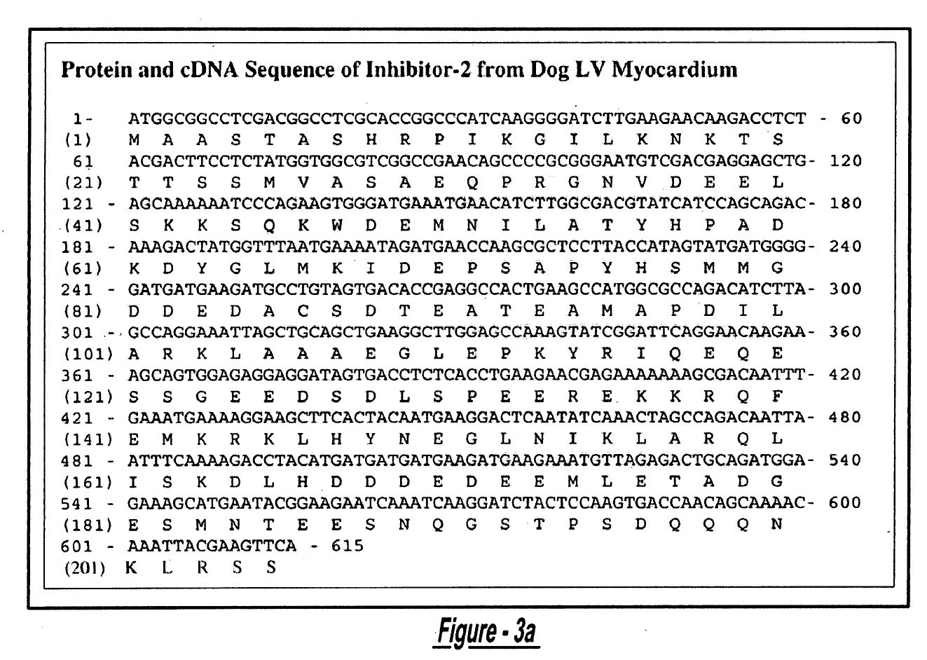

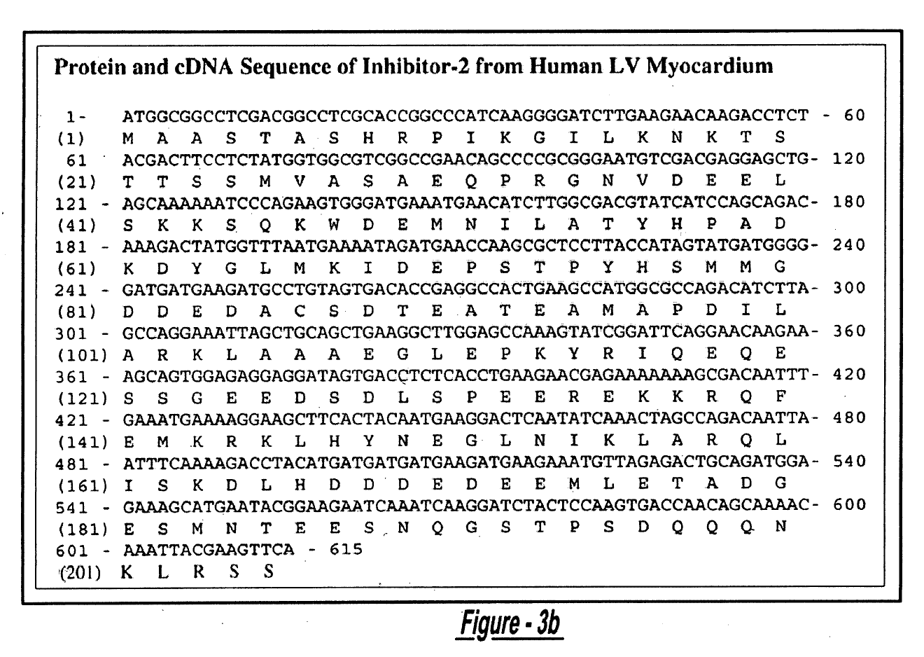

Cloning and Sequencing of I-2 DNA: Using forward and reverse primers, RNA isolated from normal dog LV myocardial tissue was reverse-transcribed into cDNA and them amplified. The product of 0.612 kb was recognized on 2% agarose gel after ethidium bromide staining as shown in FIG. 1. I-2 cDNA was then ligated into a TOPO vector and then transformed in competent TOP 10F′, bacterial strain. Four colonies were picked up. The presence of the I-2 insert in forward direction was confirmed by Hind III restriction enzyme digestion. Results are shown in FIG. 2. Hind III generates 3.12 kb and 0.27 kb digestions products from the clone having reverse direction. Only clone 1 and 2 had the inhibitor-2 insert in forward direction, clone 3 had reverse direction, and clone 4 had no insert (FIG. 2). Plasmids from clones 1 and 2 were isolated and the insert was then sequenced. Results are shown in FIG. 3. Sequences of both clones were found to be in forward directions. Nucleotide sequences of dog heart I-2 has approximately 99% homology with that of human colon and lymphocyte, 91% with rat brain, and 86% with rabbit skeletal muscle. The predicted amino acid sequence of dog heart I-2 was very similar to that of human colon and lymphocyte except alanine at position 73 in human was changed in threonine in dog heart I-2(FIG. 3).

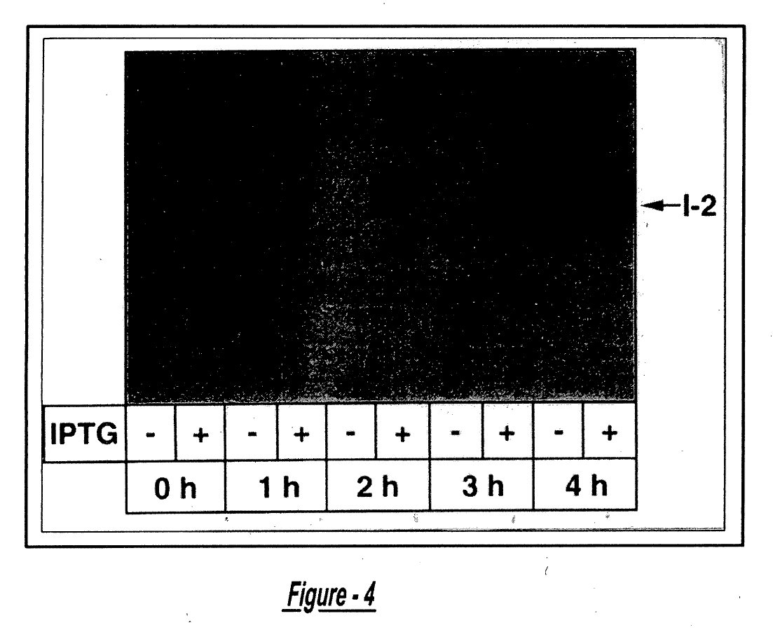

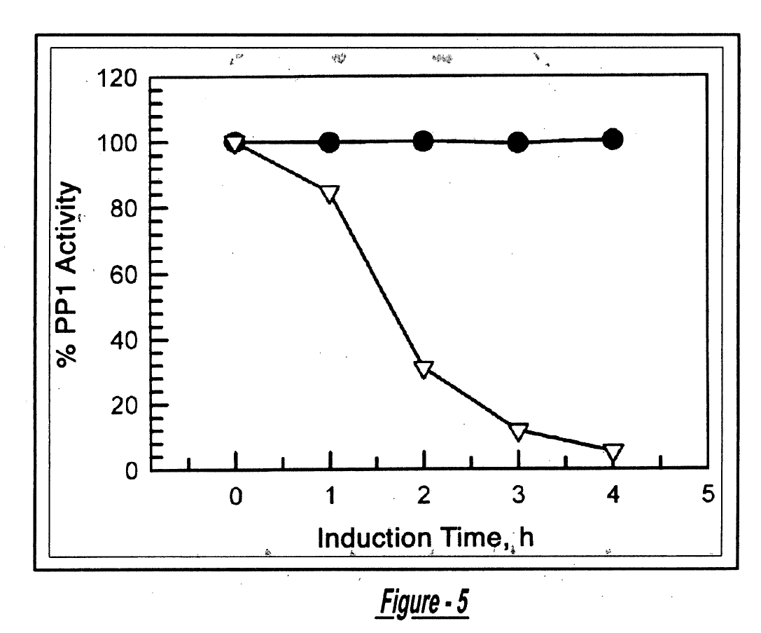

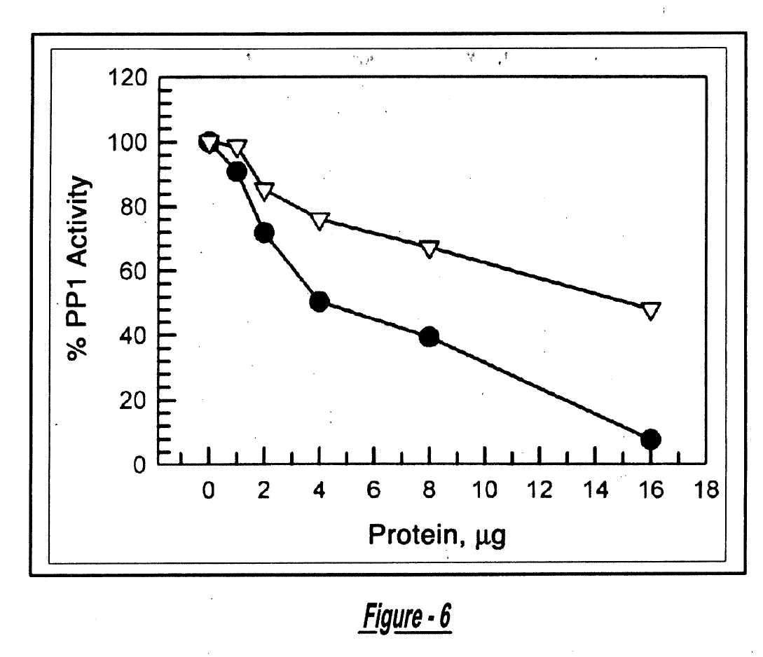

Inhibitor-2 Gene Expression: To express I-2 cDNA, plasmid from clone 1 or 2 was transformed into gene expressing E. coli strain and the recombinant was picked up. I-2 cDNA gene was induced by 1 mM IPTG at different time points varying from 0 hour to 4 hours. The recombinant protein identity was confirmed by using I-2 specific monoclonal antibody and results are shown in FIG. 4. In the absence of IPTG, no protein was detected, whereas in the presence of IPTG protein expression increased with time up to four hours (FIG. 4). At 20 hours, no expression of I-2 was observed. In order to be sure that recombinant I-2 is biologically active i.e. it inhibits PP1 activity, the effect of the bacterial homogenate at different time points was examined on PP1 activity. Results are shown in FIG. 5. Only the bacterial extract in the presence of IPTG, but not in the absence of IPTG, inhibited the phosphatase activity (FIG. 5). To gain further insight, bacterial homogenate was separated into cytosolic and membrane fractions and then their effects were monitored on phosphatase activity. Both the membrane and cytosol inhibited PP1 activity, but the extent of inhibition was approximately two fold higher in the cytosol than membrane suggesting that the presence of the recombinant protein is about two fold higher in the cytosol than membrane (FIG. 6).

In summary, dog cardiac I-2 cDNA has been isolated, cloned, and expressed in E. coli. This clone expresses I-2 protein, which was confirmed by immunoblotting and by its ability to inhibit PP1 activity, a characteristic observed for the purified rabbit skeletal muscle I-2. Sequence of cardiac I-2 cDNA is very similar to that reported for human colon except threonine 73 in human colon changed to alanine 72 in cardiac 1-2.

Heart failure affects over 5 million people in the United States alone. Despite aggressive therapy, most, if not all patients succumb to the disease. The need for novel therapeutic modalities that address the progressive LV dysfunction that leads to intractable heart failure is paramount. The present approach of treating heart failure by selectively inhibiting cardiac type-1 protein phosphatase through overexpression of cDNA encoding for I-2 has not been suggested previously and can overcome the problems set forth previously.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

### REFERENCES

Burke and Olson, “Preparation of Clone Libraries in Yeast Artificial-Chromosome Vectors” in Methods in Enzymology, Vol. 194, “Guide to Yeast Genetics and Molecular Biology”, eds. C. Guthrie and G. Fink, Academic Press, Inc., Chap. 17, pp. 251-270(1991).

Capecchi, “Altering the genome by homologous recombination” Science 244:1288-1292 (1989).

Davies et al., “Targeted alterations in yeast artificial chromosomes for inter-species gene transfer”, Nucleic Acids Research, Vol. 20, No. 11, pp. 2693-2698 (1992).

Dickinson et al., “High frequency gene targeting using-insertional vectors”, Human Molecular Genetics, Vol. 2, No. 8, pp. 1299-1302 (1993).

Duff and Lincoln, “Insertion of a pathogenic mutation into a yeast artificial chromosome containing the human APP gene and expression in ES cells”, Research Advances in Alzheimer's Disease and Related Disorders, 1995.

Huxley et al., “The human HPRT gene on a yeast artificial chromosome is functional when transferred to mouse cells by cell fusion”, Genomics, 9:742-750 (1991).

Jakobovits et al., “Germ-line transmission and expression of a human-derived yeast artificial chromosome”, Nature, Vol. 362, pp. 255-261 (1993).

Lamb et al., “Introduction and expression of the 400 kilobase precursor amyloid protein gene in transgenic mice”, Nature Genetics, Vol. 5, pp. 22-29 (1993).

Pearson and Choi, Expression of the human b-amyloid precursor protein gene from a yeast artificial chromosome in transgenic mice. Proc. Natl. Acad. Sci. USA, 1993. 90:10578-82.

Rothstein, “Targeting, disruption, replacement, and allele rescue: integrative DNA transformation in yeast” in Methods in Enzymology, Vol. 194, “Guide to Yeast Genetics and Molecular Biology”, eds. C. Guthrie and G. Fink, Academic Press, Inc., Chap. 19, pp. 281-301 (1991).

Schedl et al., “A yeast artificial chromosome covering the tyrosinase gene confers copy number-dependent expression in transgenic mice”, Nature, Vol. 362, pp. 258-261 (1993).

Strauss et al., “Germ line transmission of a yeast artificial chromosome spanning the murine a₁ (I) collagen locus”, Science, Vol. 259, pp. 1904

Gilboa, E, Eglitis, MA, Kantoff, PW, Anderson, WF: Transfer and expression of cloned genes using retroviral vectors. BioTechniques 4(6):504-512, 1986.

Cregg JM, Vedvick TS, Raschke WC: Recent Advances in the Expression of Foreign Genes in Pichia pastoris, Bio/Technology 11:905-910,1993

Culver, 1998. Site-Directed recombination for repair of mutations in the human ADA gene. (Abstract) Antisense DNA & RNA based therapeutics, February, 1998, Coronado, Calif.

Huston et al, 1991 “Protein engineering of single-chain Fv analogs and fusion proteins” in Methods in Enzymology (JJ Langone, ed.; Academic Press, New York, N.Y.) 203:46-88.

Johnson and Bird, 1991 “Construction of single-chain Fvb derivatives of monoclonal antibodies” and their production in Escherichia coli in Methods in Enzymology (JJ Langone, ed.; Academic Press, New York, N.Y.) 203:88-99.

Mernaugh and Mernaugh, 1995 “An overview of phage-displayed recombinant antibodies” in Molecular-Methods In Plant Pathology (RP Singh and US Singh, eds.; CRC Press Inc., Boca Raton, Fla.) pp. 359-365.

What is claimed is:

1. A method for treating an individual with cardiovascular disease, comprising the step of administering a therapeutically effective amount of protein phosphatase inhibitor and analogues thereof, to said individual after the onset of cardiac ischemia.

2. The method of claim 1, wherein said individual is having a myocardial infarction.

3. A method of treating an individual with cardiovascular disease by selectively inhibiting protein phosphatase activity in the heart.

4. The method according to claim 3, wherein said inhibiting step includes administering an effective amount of a protein phosphatase inhibitor.

5. A composition for the treatment of heart failure, said composition comprising an inhibitor of PP1 and a pharmaceutically acceptable carrier.

6. The composition according to claim 5, wherein said inhibitor is inhibitor-2.

7. A sequence encoding inhibitor-2.

8. The sequence according to claim 7, wherein said sequence is isolated from humans.

9. The sequence according to claim 7, wherein said sequence is isolated from dogs.

10. The sequence according to claim 7, wherein said sequence is selected from the group consisting essentially of SEQ ID Nos: 1 and 2.


SEQUENCE LISTING (4 sequences)

SEQ ID NO: 1 - protein, 205 aa, Canine [sequence omitted]

SEQ ID NO: 2 - protein, 205 aa, Homo sapien
MAASTASHRPIKGILKNKTSTTSSMVASAEQPRGNVDEELSKKSQKWDEMNILATYHPADKDYGLMKIDEPSTPYHSMMGDDEDACSDTEATEAMAPDILARKLAAAEGLEPKYRIQEQESSGEEDSDLSPEEREKKRQFEMKRKLHYNEGLNIKLARQLISKDLHDDDEDEEMLETADGESMNTEESNQGSTPSDQQQNKLRSS

SEQ ID NO: 3 - DNA, 24 bp, Homo sapien
ccaatggcgggcctcgacggcctc

SEQ ID NO: 4 - DNA, 26 bp, Homo sapien
tcgtaatttgtttgctgttggtcact